(19) United States
(12) Patent Application Publication
(10) Pub. No.: US 2022/0313728 A1
(43) Pub. Date: Oct. 6, 2022

(54) COMPOSITIONS AND METHODS FOR TREATING TREATMENT-RESISTANT DEPRESSIVE DISORDERS WITH NITROUS OXIDE

(71) Applicant: Peter Nagele, Chicago, IL (US)

(72) Inventor: Peter Nagele, Chicago, IL (US)

(21) Appl. No.: 17/657,749

(22) Filed: Apr. 2, 2022

Related U.S. Application Data
(60) Provisional application No. 63/200,909, filed on Apr. 2, 2021.

(51) Int. Cl. A61K 33/00; A61P 25/24

(52) U.S. Cl. CPC A61K 33/00; A61P 25/24

(57) ABSTRACT

The present invention generally relates to the use of 25% nitrous oxide for treating patients with a treatment-resistive depressive disorder and compositions useful for the same.

### FIELD OF THE INVENTION

The present invention generally relates to the use of 25% by weight nitrous oxide for treating patients with treatment-resistant depressive disorder and compositions useful for the same.

### BACKGROUND OF THE INVENTION

Treatment-resistant depression is a particularly severe form of major depressive disorder. Affecting one in three patients with major depressive disorder (estimated prevalence in the United States is 10 million adults), patients with treatment-resistant depression often fail multiple treatments with standard antidepressants and have an unfavorable long-term prognosis. Therapeutic options for treatment-resistant depression are scarce.

In WO2015175531 it was described how nitrous oxide (laughing gas)(50% by weight, inhaled concentration) improved depressive symptoms in patients with treatment-resistant major depression (TRMD)(one dosage, with results reported for up to 1 week post dosing). The use of nitrous oxide, 50% by weight, was not commercially advanced.

Thus, there remains an unmet need for treatments for depression that is only partially responsive to medication and intractable (e.g., treatment-resistant).

### SUMMARY OF THE INVENTION

In an aspect, the present invention provides a novel method of treating a treatment-resistant depressive disorder in a subject, comprising administering to the subject an inhaled gas, comprising 25% by weight of nitrous oxide.

In another aspect, the present invention provides a novel, isolated composition, comprising: an inhalable gas, comprising: 25% by weight of nitrous oxide.

In another aspect, the present invention provides a novel, isolated composition for use in medical therapy.

In another aspect, the present invention provides the use of novel, isolated compositions of the present invention for the manufacture of a medicament for the treatment of a treatment-resistant depressive disorder in a subject.

These and other aspects, which will become apparent during the following detailed description, have been achieved by the inventors' discovery of a novel method of treating a depressive disorder in a subject using 25% by weight nitrous oxide that now has been shown to have unexpectedly similar efficacy to 50% nitrous oxide with unexpectedly and substantially reduced side effects.

### BRIEF DESCRIPTION OF THE DRAWINGS

FIG. 1 shows the relative change in depressive symptoms between 50% nitrous oxide, 25% nitrous oxide, and placebo on the Hamilton Depression Rating Scale (primary outcome)(means+/−95% confidence interval).

FIG. 2 shows a comparison of the severity of depressive symptoms before and after study completion (means+/−95% confidence interval).

FIG. 3 shows the proportion of patients who experienced response or remission (based on HDRS-21) after treatment with 50% nitrous oxide, 25% nitrous oxide, and placebo.

### DETAILED DESCRIPTION OF THE PREFERRED ASPECTS

Exemplary aspects of the present invention are described with reference to the figures, where appropriate. Although the following detailed description contains many specifics for purposes of illustration, a person of ordinary skill in the art will appreciate that variations and alterations to the following details are within the scope of the invention. Accordingly, the following aspects of the invention are set forth without any loss of generality to, and without imposing limitations upon, the claimed invention.

Abbreviations and Definitions

When introducing elements of the present disclosure or an aspect thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

The terms “depression” or “depressive disorder” refers to any nervous system disorder and/or mental condition characterized by the following symptoms: depressed mood, anhedonia, feelings of intense sadness and despair, mental slowing, loss of concentration, pessimistic worry, agitation, self-deprecation, disturbed sleep patterns (e.g. insomnia, loss of REM sleep, or hypersomnia), anorexia, changes in appetite and weight loss or weight gain, psychomotor agitation, decreased energy, decreased libido, and changes in hormonal circadian rhythms, withdrawal, altered daily rhythms of mood, activity, temperature, and neuroendocrine function, and combinations thereof. Non-limiting examples of “depression” include major depressive disorder, bipolar depressed mood disorder, adjustment mood disorder, and post-partum mood disorder.

The terms “treatment,” “treating” or “treat,” when referring to a condition, and as understood in the art, are defined to mean an approach for obtaining beneficial or desired results, including clinical results. Beneficial or desired clinical results can include alleviation of one or more symptoms of the condition, diminishment of extent of disease or condition, stabilized (i.e., not worsening) state of disease or condition, preventing spread of disease, delay or slowing of disease progression, palliation of the disease state, and remission (whether partial or total), whether detectable or undetectable.

The terms “subject” or “patient” are used interchangeably and mean all members of the animal kingdom (e.g., humans).

The term “subject in need thereof” when referring to nitrous oxide administration, means a subject having a condition that can be treated with nitrous oxide.

The term “effective amount” or “pharmaceutically effective amount” are used interchangeably and are defined to mean the amount or quantity of nitrous oxide, which is sufficient to elicit an appreciable biological response when administered to a patient. It will be appreciated that the precise therapeutic dose will depend on the age and condition of the patient and the nature of the condition to be treated and will be at the ultimate discretion of the attendant physician.

BRPS-18 refers to the Brief Psychiatric Rating Scale 18-item.

CADSS-28 refers to the Clinical Administered Dissociative States Scale 28-item

DSM-IV refers to the Diagnostic and Statistical Manual of Mental Disorders.

HDRS refers to the Hamilton Depression Rating Scale (HDRS-21, the 21 item scale) (HDRS-17, the 17 item scale).

IQR refers to the interquartile range.

MADRS refers to the Montgomery-Asberg Depression Rating Scale.

MDD refers to major depressive disorder.

MINI refers to the Mini International Neuropsychiatric Interview.

NMDA refers to N-methyl-D-aspartic acid.

POMS-2 refers to the Profile of Mood States 2^{nd} Edition.

QIDS-SR refers to the Quick Inventory of Depressive Symptomology-Self Report.

RR refers to relative risk.

rTMS refers to repetitive transcranial magnetic stimulation.

SSRI refers to selective serotonin reuptake inhibitor.

SNRI refers to serotonin—norepinephrine reuptake inhibitor.

TRMD refers to treatment-resistant major depression.

Methods and Compositions

In an aspect, the present invention provides a novel method of treating a treatment-resistant depressive disorder in a subject in need thereof, comprising: administering to the subject an effective amount of an inhaled gas, comprising: 25% by weight of nitrous oxide.

In another aspect, the treatment-resistant depressive disorder is selected from atypical depression, bipolar disorder, catatonic depression, depressive disorder not otherwise specified, depressive personality disorder, double depression, dysthymia, major depressive disorder, melancholic depression, minor depressive disorder, postpartum depression, post-traumatic stress disorder, psychotic major depression, recurrent brief depression, seasonal affective disorder, suicidality/acute suicide risk, and treatment-resistant major depression.

In another aspect, the depressive disorder is treatment-resistant major depression (TRMD).

In another aspect, the subject is human.

In another aspect, the inhaled gas, further comprises: oxygen, nitrogen, xenon, or combinations thereof.

In another aspect, the inhaled gas, comprises: 25% by weight nitrous oxide, 5-25% by weight xenon, and the remainder oxygen.

In another aspect, the inhaled gas, comprises: 25% by weight nitrous oxide and, 5-25% by weight nitrogen, and the remainder oxygen.

In another aspect, the inhaled gas, comprises: 25% by weight nitrous oxide and 75% oxygen.

In another aspect, the inhaled gas is administered at a flow rate of 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5 to 10.0 liters per minute (L/min). Examples include 1-9 L/min and 2-8 L/min.

In another aspect, the inhaled gas is administered for 1, 5, 10, 15, 20, 25, 30, 35, 40, 45, 50, 55, 60, 65, 70, 75, 80, 85, to 90 minutes. Examples include 15-60 minutes, 30-60 minutes, and 60 minutes.

In another aspect, when nitrous gas (e.g., −100% nitrous oxide) is mixed with a carrier gas (e.g., oxygen, nitrogen/oxygen, xenon/oxygen, air, or a combination thereof) prior to inhalation (to titrate the mixture of carrier gas and nitrous oxide from 0 to 25% by weight nitrous oxide), the total treatment time includes titrating the nitrous oxide to 25% by weight. Examples of the titration time include from 1, 2, 3, 4, 5, 6, 7, 8, 9, 10, 11, 12, 13, 14, to 15 minutes. Another example includes 5-10 minutes.

In another aspect, the inhaled gas is administered at least one day every seven days of treatment. Examples include every day, every other day, every third day, every fourth day, every fifth day, and every sixth day of a treatment period. Other examples include once every two weeks of treatment, once every three weeks of treatment, and one every four weeks of treatment.

In another aspect, the treatment period is for at least 1, 2, 3, to 4 weeks. Examples include at least 1, 2, 3, 4, 5, 6, 7, 8, 9, 10, 11, to 12 months. Other examples include at least 0.5, 1, 15, 2, 2.5, to 3 years.

In another aspect, the present invention provides a novel method of treating a treatment-resistant depressive disorder in a subject in need thereof, comprising: (a) administering to the subject an effective amount of an inhaled gas, comprising: 25% by weight of nitrous oxide for a first treatment period, and (b) administering to the subject an effective amount of an inhaled gas, comprising: 50% by weight of nitrous oxide for a second treatment period. This dose escalation treatment would typically be used when a stronger treatment effect is desired (e.g., after observing the effects of the 25% nitrous treatment).

In another aspect, the first treatment period is for at least 1, 2, 3, to 4 weeks. Examples of the first treatment period include at least 1, 2, 3, 4, 5, 6, 7, 8, 9, 10, 11, to 12 months.

In another aspect, the second treatment period is for at least 1, 2, 3, to 4 weeks. Examples of the second treatment period include at least 1, 2, 3, 4, 5, 6, 7, 8, 9, 10, 11, to 12 months. Other examples of the second treatment period include at least 0.5, 1, 15, 2, 2.5, to 3 years.

In another aspect, the present invention provides a novel, isolated, inhalable gas, comprising: 25% by weight nitrous oxide. The inhalable gas is typically housed (isolated) in a gas container. The gas container is one that is suitable to both store the inhalable gas (for long term storage and transportation) as well as be connected (typically via a valve) to a device suitable to deliver the gas to a subject being treated. An example of a gas container is a gas cylinder or gas bottle that is suitable for storing the inhalable gas at a pressure above (or well above) atmospheric pressure. In another aspect, the gas container, comprises: a gas valve configured for filling and controlling the rate of gas escaping from the container. In another aspect, the gas container, further comprises: a pressure gauge configured to display the pressure of inhalable gas in the gas container. In another aspect, the valve and pressure gauge are separate but operably connected (e.g., the gauge is threaded onto the valve via a coupling). The volume of the gas container can vary based on the intended use (and frequency thereof). Examples of the gas container volume (internal volume at 21° C., 1 atm) include 1, 2, 3, 4, 5, 6, 7, 8, 9, 10, 15, 20, 25, 30, 35, 40, 45, 50, 55, 60, 65, 70, 75, 80, 85, 90, 95, to 100 L.

In another aspect, the present invention provides a compound for use in therapy.

In another aspect, the present invention provides the use of compounds for the manufacture of a medicament for the treatment of an indication recited herein.

The present invention may be embodied in other specific forms without departing from the spirit or essential attributes thereof. This invention encompasses all combinations of aspects of the invention noted herein. It is understood that any and all embodiments of the present invention may be taken in conjunction with any other embodiment or embodiments to describe additional embodiments. It is also to be understood that each individual element of the embodiments is intended to be taken individually as its own independent embodiment. Furthermore, any element of an embodiment is meant to be combined with any and all other elements from any embodiment to describe an additional embodiment.

### EXAMPLES

Summary of Clinical Results

Nitrous oxide (at 50% inhaled concentration) has been shown to improve depressive symptoms in patients with treatment-resistant major depression (TRMD). At the time of the study, it was unknown whether a lower concentration of nitrous oxide (25%) would provide similar efficacy and persistence of antidepressant effects while reducing the risk of adverse side effects. In the phase 2 clinical trial described herein, 24 patients with severe TRMD were randomly assigned in a crossover fashion to 3 treatments consisting of a single 1-hour inhalation with (1) 50% nitrous oxide, (2) 25% nitrous oxide, or (3) placebo (air/oxygen). Primary outcome was the change on the Hamilton Depression Rating Scale (HDRS-21). Nitrous oxide significantly improved depressive symptoms versus placebo (p=0.01) and there was no statistically significant difference between 25% and 50% nitrous oxide (p=0.58).

The estimated differences between 25% and placebo were −0.75 points (HDRS-21) at 2 hours (p=0.73), −1.41 points at 24 hours (p=0.52), −4.35 points at week 1 (p=0.05), and −5.19 points at week 2 (p=0.02). The estimated differences between 50% and placebo were: −0.87 points at 2 hours (p=0.69), −1.93 points at 24 hours (p=0.37), −2.44 points at week 1 (p=0.25), and −7.00 points at week 2 (p=0.001). Adverse events declined with dose: 47 (50% nitrous oxide), 11 (25% nitrous oxide), and 6 (placebo) (p<0.001). These results suggest that 25% nitrous oxide has comparable efficacy to 50% nitrous oxide in improving TRMD but with a markedly lower rate of adverse effects.

Background

Treatment-resistant major depression (TRMD) is a severe form of major depressive disorder (MDD) in which patients fail to respond to multiple standard antidepressant treatments. (1, 2) The lifetime prevalence of major depressive disorder is estimated to be approximately 10-20%, of which at least one third of patients are estimated to be at risk for TRMD. (3-5) For the US alone, this equates to approximately 17 million adults with TRMD. (6)

A proof-of-principle study demonstrated that a one-hour inhalation of 50% nitrous oxide (“laughing gas”) has rapid antidepressant effects in patients with TRMD. (7) The study had two important limitations. First, it did not formally test whether antidepressant effects lasted beyond 24 hours. Second, it used a high concentration of nitrous oxide (50%), for which risk of nausea and other unwanted side effects may limit its clinical use. Moreover, evidence from the antidepressant use of ketamine, (8) a drug with a similar proposed mechanism of action (NMDA-receptor antagonism), (9-11) suggests that a lower subanesthetic dose may be equally efficacious while potentially conferring a lower risk of side effects. (12-14)

In this trial, the goal was to determine whether a lower concentration of nitrous oxide (25%) had comparable antidepressant efficacy in TRMD as 50% nitrous oxide. Additional goals of this investigation were to determine whether inhalation of 25% nitrous oxide would be associated with fewer side effects, and whether the antidepressant effects of nitrous oxide would extend beyond 24 hours after a single inhalation treatment with a follow-up period of at least 14 days.

Methods

Study Design and Oversight

The study was a single-center, double-blind, randomized placebo-controlled crossover trial. All subjects underwent three one-hour inhalation sessions in random order, each separated by at least 4 weeks. The sessions included: placebo (0% N20), 25% N20, and 50% N20 balanced with air/oxygen. Blinding was executed by separating locations and teams for inhalation treatments and psychiatric evaluations. Only the anesthesia team administering the inhalation treatments was aware of study group assignment; all other participants, including patients and raters, were blinded. Likewise, the study setup was identical for all sessions, and gas flow meters were concealed, making inadvertent unblinding unlikely. The study was approved by the Washington University in St. Louis Institutional Review Board, and all patients provided written, informed consent. The trial was registered at clinicaltrials.gov (NCT03283670).

Patients

Patients were recruited from an existing database of TRMD patients identified through participation in a secondary referral clinic for TRMD at Washington University Department of Psychiatry, as well as from the “Volunteers for Health” patient pool (individuals with various medical/psychiatric conditions who volunteer to participate in clinical research) within Washington University School of Medicine. Inclusion criteria were a) adults 18-75 years of age; b) current diagnosis of unipolar major depressive disorder (MDD) without psychosis as confirmed by the Mini International Neuropsychiatric Interview; c) a score of ≥19 on the Montgomery-Asberg Depression Rating Scale (MADRS); d) documented lifetime failure to respond to ≥3 adequate dose/duration antidepressant treatment trials, including ≥1 antidepressant medication failure(s) in the current depressive episode; and e) good command of the English language. Exclusion criteria were: a) meeting criteria for any Diagnostic and Statistical Manual of Mental Disorders (DSM-IV) diagnosis for schizophrenia, bipolar, schizoaffective, obsessive-compulsive, personality, or panic disorders; b) any recent (within past 12 months) history of substance dependence or abuse (except tobacco), determined by reported history and urine drug screen; c) ability to become pregnant and not using effective contraception; d) contraindication against the use of nitrous oxide; e) inability to provide informed consent; and f) any other factor that in the investigators' judgment may affect patient safety or compliance. Patients were instructed to continue their current standard of care MDD treatment and to maintain a stable psychotropic medication dosage or psychotherapy regimen for 4 weeks prior to initiation of the study and throughout the study. Further, patients were instructed not to modify their antidepressant treatments during the three month course of the trial (i.e., discouraged from adding new antidepressants or modifying existing antidepressant dosages).

Study Procedures

There were a total of 14 planned study visits for each patient. A screening visit was used to verify eligibility and to collect background information including patient demographics, medical history, vital signs, and a physical exam. A urine sample was collected for a drug screen, and an optional blood draw was collected if the study physician requested tests to confirm patients' safety to participate. A structured clinical interview, the Mini International Neuropsychiatric Interview (MINI), (15) and other psychiatric assessments measuring baseline depression severity were completed by the research team and patients. This rigorous screening verified the appropriate primary diagnosis and ruled out excluded diagnoses (e.g., post-traumatic stress disorder, psychotic disorders, severe comorbid personality disorders, and substance use disorders).

Following the in-person screen, each of the three treatment sessions consisted of four visits: pre-inhalation mood assessment, inhalation, and post-inhalation follow-up sessions at 22-28 hours, 1 week, and 2 weeks. An additional assessment was completed 4 weeks following the final inhalation treatment.

Inhalation Sessions

All three inhalation sessions were scheduled for one hour. Patients received an admixture of (1) placebo (air/oxygen), (2) 25% nitrous oxide in oxygen, or (3) 50% nitrous oxide in oxygen. Except for the choice of inhalational gas admixture, treatment sessions were otherwise identical. The gas mix was administered via a standard anesthesia facemask through tubing connected to the anesthesia machine or an FDA-approved Porter/Praxair MXR breathing circuit. A small sample connector line was inserted into the facemask allowing the measurement of inhaled and exhaled gas concentrations. Total gas flow was 2-8 L/min and nitrous oxide concentrations were gradually titrated upwards over the course of the first 5-10 minutes of treatment. Patients were monitored during and after the treatment according to American Society of Anesthesiologists standards which include continuous 3-lead ECG, pulse oximetry, non-invasive blood pressure, and end tidal CO2 measurement under the supervision of an attending-level anesthesiologist. After the one-hour treatment session, patients were monitored in a recovery room for up to one hour at which time a study team physician determined whether the patient met criteria for discharge.

Outcomes

Data were collected at (a) baseline (prior to each inhalation session), (b) 2-hours, (c) 24 hours, (d) 1 week, and (e) 2 weeks after inhalation. Efficacy of the inhalation treatment on mood was measured using the following: Hamilton Depression Rating Scale 21-item (HDRS-21; primary outcome); Montgomery-Asberg Depression Rating Scale (MADRS); Quick Inventory of Depressive Symptomology-Self Report (QIDS-SR); and the Profile of Mood States 2nd Edition (POMS-2). Other behavioral assessments included an assessment of dissociation, the Clinical Administered Dissociative States Scale 28-item (CADSS-28); and an assessment for the emergence of psychosis, the Brief Psychiatric Rating Scale 18-item (BPRS-18). These mood and behavioral assessments were performed by a blinded member of the psychiatry study team. Additionally, adequacy of the blind was assessed by a Blinding Questionnaire given after completion of each inhalation session. Treatment safety was assessed by monitoring adverse events (AEs) related to (a) cardiovascular status; (b) respiratory function; (c) central nervous system; and (d) psychiatric symptoms, particularly the presence of psychotic symptoms.

Statistical Analysis

The intention-to-treat analysis was based on mixed-effects linear regression models (Hedeker and Gibbons, 2006), to accommodate correlation produced by the within-subject cross-over design. The primary analysis included treatment group (placebo, 25% and 50%), time (baseline, 2-hours, 24 hours, 1 week, and 2 weeks after inhalation), period, and the treatment by time interaction as categorical variables (random intercept model). The period effect adjusts for the cumulative effect of treatment (in random orders) over the course of the study. The treatment by time interaction tests the null hypothesis of no difference between the treatment groups in the severity of depressive symptoms (HDRS-21) over time using a likelihood ratio chi-square statistic. The same analysis was performed to directly compare the 25% versus 50% treatment groups to each other, and the combined 25% and 50% dosage groups to placebo. These models also provide point in time between group comparisons using Wald tests. A comparison of linear time trends between each of the active treatment groups and placebo was also performed.

Finally, we also tested for a linear dose-response relation. Differences between categorical outcomes (response, remission) were tested by Fisher-Exact test; relative risk and 95% confidence intervals were calculated using the Koopman asymptotic score. Differences in the count of adverse events were compared by the Kruskal-Wallis test. All reported p-values are two-sided and a p-value of <0.05 was considered statistically significant. All analyses were conducted using SuperMix (Scientific Software International, Lincolnwood Ill.).

Results

Patients

After excluding 6 patients for screen failure, 28 patients were enrolled between November 2016 and October 2019. Twenty patients completed all three inhalation sessions (placebo, 25% nitrous oxide, 50% nitrous oxide); one patient withdrew after two sessions, three patients after one session, and four patients withdrew after screening but before any treatment. Results are reported from the 20 patients who completed all 3 inhalation sessions (60 total inhalation sessions) and the 4 patients who received at least one treatment. One patient inadvertently received two sessions with 50% nitrous oxide and no placebo, resulting in a final number of 22 treatments with placebo, 20 treatments with 25% nitrous oxide, and 23 treatments with 50% nitrous oxide.

Study patients had sustained and highly refractory depressive illness with an average of 17.5 lifetime years of MDD and having failed a median of 4.5 (interquartile range (IQR) 3-10) adequate-dose/duration antidepressant drug treatments (Table 1). The median HDRS-21 score at enrollment was 20.5 and the median MADRS score was 30, indicative of severe TRMD.

TABLE 1
Baseline Characteristics (N = 24)

| | |
|---|---|
| Age-yrs | 39 [26 - 68] |
| Female Sex-no. (%) | 17 (71%) |
| Race-no. (%) | |
| White | 23 (96) |
| Depression history-yrs | 17.5 [1-53] |
| Number of failed past treatment trials | 4.5 [3-10] |
| Baseline HDRS-21 score | 20.5 [19-25.5] |
| Baseline MADRS score | 30 [26.3-35.8] |
| Vagus nerve stimulator-no. (%) | 1 (4) |
| History of ECT-no. (%) | 2 (8) |
| History of rTMS-no. (%) | 3 (13) |
| History of ketamine-no. (%) | 2 (8) |
| History of migraines-no. (%) | 11 (46) |
| Number of current antidepressant | 1 [0-4] |
| medications | |
| Antidepressants-no. (%) | |
| SSRI | 9 (38) |
| SNRI | 6 (25) |
| Bupropion | 7 (29) |
| Tricyclic | 2 (8) |
| Atypical Antidepressants-no. | |
| (%) | |
| Mirtazapine | 1 (4) |
| Quetiapine (Seroquel) | 2 (8) |
| Trazodone | 1 (4) |
| Vilazodone | 1 (4) |
| Vortioxetine | 1 (4) |
| Other-no. (%) | |
| Lurasidone | 1 (4) |
| Lamotrigine | 2 (8) |
| Naltrexone | 1 (4) |
| Lithium | 1 (4) |
| Xanax | 1 (4) |
| Lorazepam | 1 (4) |

Numbers are listed as medians and interquartile range (IQR) or counts and percentages. rTMS-repetitive transcranial magnetic stimulation. SSRI-selective serotonin reuptake inhibitor; SNRI-serotonin—norepinephrine reuptake inhibitor.

Study Outcomes

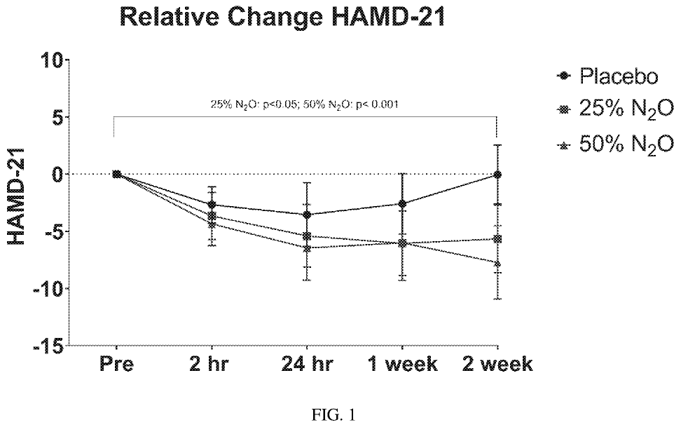

In the intention-to-treat analysis (n=24, 20 completers, 4 partial completers), the overall effect of nitrous oxide (both groups) compared to placebo on the primary outcome (HDRS-21) over the course of two weeks of observation was significant (p=0.01), but there was no significant difference between 25% and 50% nitrous oxide (p=0.58). The estimated differences between 25% and placebo were −0.75 points (HDRS-21) at 2 hours (p=0.73, d=0.16), −1.41 points at 24 hours (p=0.52, d=0.21), −4.35 points at week 1 (p=0.05, d=0.38), and −5.19 points at week 2 (p=0.02, d=0.62). The estimated differences between 50% and placebo were −0.87 points at 2 hours (p=0.69, d=0.29), −1.93 points at 24 hours (p=0.37, d=0.32), −2.44 points at week 1 (p=0.25, d=0.35), and −7.00 points at week 2 (p=0.001, d=0.85) (see FIG. 1). The estimated differences between 50% and 25% were 0.11 points at 2 hours (p=0.96), 0.42 points at 24 hours (p=0.85), 1.91 points at week 1 (p=0.37), and −1.67 points at week 2 (p=0.44). The estimated differences between placebo and the combined 25% and 50% groups were −0.81 points at 2 hours (p=0.66), −1.67 points at 24 hours (p=0.37), −3.35 points at week 1 (p=0.07), and −6.13 points at week 2 (p=0.001). Relative to placebo, the effects of the active treatment groups linearly increased over time for the 25% group (−1.38 per measurement occasion, p=0.007) and for the 50% group (−1.55 per measurement occasion, p=0.002). A significant dose-response relation was found at week 2 (a −3.51 decrease per 25% increase in dose, p=0.001), but not at earlier measurement times. To study carryover effects we performed an analysis to determine if order of receipt of the 50% dose was related to the 2 week HDRS-21 score. No significant effect trial order was found (p=0.22).

The MADRS data are similar in efficacy to the HDRS-21 results regarding 50% nitrous oxide but not for 25% nitrous oxide (not significant). Results on the QIDS scale are, except for 50% nitrous oxide at 2 weeks, not statistically significant. Results on the POMS scale show a stronger response at 50% nitrous oxide (which is significant at 2 weeks) but not for 25% nitrous oxide.

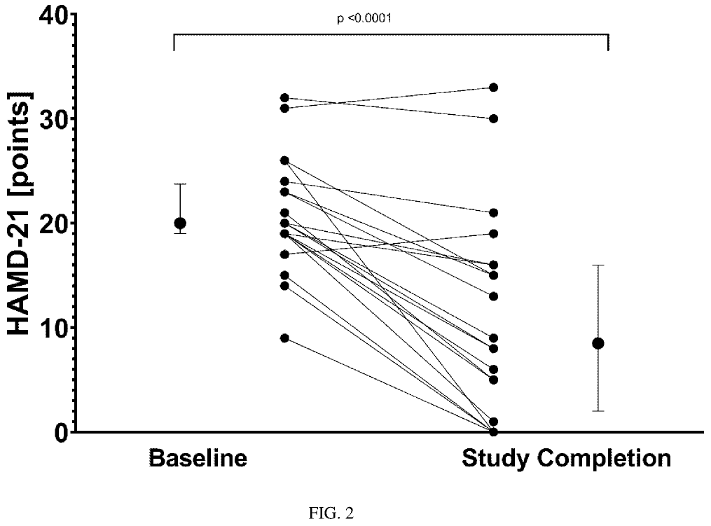

Over the entire course of treatment (including only patients who completed the study, n=20), patients experienced a clinically significant improvement in depressive symptoms from a median baseline HDRS-21 score of 20.5 (IQR 19.0-25.5) to 8.5 (IQR 2.0-16.0) at study completion, corresponding to a median change of −11.0 points (IQR −3.3 to −14.0 points, p<0.0001) after the 3-month study period (FIG. 2). At study completion, 11/20 patients (55%) had a treatment response (reduction in HRDS-21 points ≥50%), 8/20 (40%) were in remission (HRDS≤7 points), and 17/20 (85%) had an improvement in depressive symptoms by at least one category (i.e., from severe to moderate).

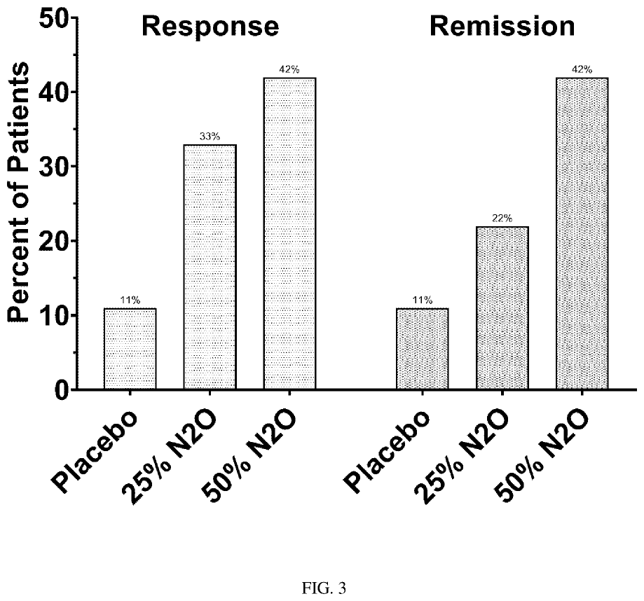

FIG. 3 demonstrates the rates of treatment response and remission for each inhalation treatment (in this analysis we only included treatments where the pre-treatment HDRS-21 score was >19). After placebo treatment, 1/9 patients had a treatment response (11.1%) and 1/9 were in remission (11.1%); after 25% nitrous oxide, 3/9 patients had a treatment response (33.3%, relative risk (RR) 2.50, 95% CI 0.43-16.30) and 2/9 were in remission (22.2%, RR 1.82, 95% CI 0.27-12.84); after 50% nitrous oxide 5/12 patients had a treatment response (41.7%, RR 2.94, 95% CI 0.57-18.02) and 5/12 were in remission (41.7%, RR 2.94, 95% CI 0.57-18.02).

We assessed the quality of blinding by including a questionnaire after each inhalation session in which patients were asked if they were receiving active treatment or placebo. In 50 of 60 sessions (83%) patients correctly guessed their treatment group, 8/60 (13%) incorrectly, and in two instances, patients did not know. Review of the records demonstrated that 4 patients either added (n=2) or increased (n=2) antidepressant dosages during the course of the study; 4 patients had confirmed antidepressant medication decreases and 2 patients had confirmed antidepressant medication discontinuation.

Safety

We observed a statistically significant difference in adverse events between treatments: 47 adverse events after inhalation with 50% nitrous oxide, 11 adverse events after inhalation with 25% nitrous oxide, and 6 after placebo inhalation (p<0.0001). None of the adverse events were serious and nearly all occurred either during or immediately after the treatment session and resolved within several hours (Table 2).

TABLE 2
Adverse Events

| | | | |
|---|---|---|---|
| | 50% N₂O | 25% N₂O | Placebo |
| Adverse Event | (n = 23) | (n = 20) | (n = 22) |

I. During or immediately after inhalation session

| | | | |
|---|---|---|---|
| Haziness | 3 (13%) | 1(5%) | 0 |
| Dizziness | 3 (13%) | 0 | 0 |
| Lightheadedness | 2 (9%) | 1 (5%) | 0 |
| Laughing | 3 (13%) | 0 | 0 |
| Feeling | 6 (26%) | 0 | 1 |
| disconnected | | | |
| Feeling high | 3 (13%) | 0 | 0 |
| Memory gaps | 1 (4%) | 0 | 0 |
| Paranoia | 1 (4%) | 0 | 0 |
| Headache | 4 (17%) | 2 (10%) | 3 (14%) |
| Sleepiness | 2 (9%) | 1 (5%) | 0 |
| Weakness/heavy | 2 (9%) | 1 (5%) | 0 |
| Nausea | 5 (21%) | 1 (5%) | 0 |
| Vomiting | 2 (9%) | 0 | 0 |
| Dry mouth | 2 (9%) | 0 | 0 |
| Tingling | 3 (13%) | 1 (5%) | 0 |

II. >24 hours after inhalation session

| | | | |
|---|---|---|---|
| Cramps | 1 (4%) | 0 | 0 |
| Sore throat | 1 (4%) | 0 | 0 |
| Intestinal gas | 0 | 1 (5%) | 0 |
| Common | 3 (13%) | 2 (10%) | 0 |
| cold/strep throat | | | |
| Stomach virus | 0 | 0 | 2 (9%) |
| Shoulder/Arm | 1 (4%) | | |
| Pain | | | |
| Car Crash | 1 (4%) | | |
| (minor) | | | |
| Fainting Spell | 1 (4%) | | |
| Total | 47 | 11 | 6 |

Discussion

In this randomized-controlled phase 2 crossover trial, we made several key observations. First, the trial extends the original findings in that a single one-hour inhalation of nitrous oxide, at either 50% or 25%, provides rapid antidepressant efficacy in patients with severe TRMD. (7) Second, the antidepressant effects increased in magnitude over time, lasting up to four weeks in some patients. Third, the overall trend demonstrated a high rate of response, remission, and symptom improvement in a population of severely TRMD depressed patients: 3 months from study initiation at completion, 85% of patients had improved, 55% had a treatment response, and 40% were in remission. Fourth, this trial found that 25% or 50% nitrous oxide have equivalent antidepressant efficacy; however, 25% nitrous oxide demonstrated a markedly lower rate of adverse side effects. Fifth, though the two dosages demonstrated roughly equivalent antidepressant efficacy, there is evidence of a dose-response relationship at the two-week follow-up. Sixth, individual time-trends show considerable inter-individual variability; however, incorporating this variability in our statistical models does not mute the significance of the treatment-related effects.

Discussion-Efficacy

Although we observed antidepressant effects in most patients after nitrous oxide inhalation, the response was not uniform. Some patients had minimal or no improvement after nitrous oxide and placebo and should be considered non-responders. Further, some patients had a strong placebo response, which in some instances mirrored the response to nitrous oxide. Of particular importance, we also observed that despite a 4-week interval between inhalation sessions, some patients showed sustained improvement of their depressive symptoms and did not return to their pre-treatment baseline level of depression severity. While inhalation with 25% nitrous oxide was statistically similar to 50%, on average there was a tendency towards greater improvement in depressive symptoms in the 50% nitrous oxide group at 2 weeks post-treatment. Additionally, the antidepressant response to nitrous oxide on the self-reported POMS scale, which measures immediate mood effects, supports that patients reported stronger efficacy of 50% as compared to 25% nitrous oxide. It is also of note that the majority of patients saw a marked improvement of their depressive symptoms throughout completion of the study where each received two nitrous oxide and one placebo treatment over the course of three months. While a Hawthorne effect (being in a clinical study) and placebo effects may explain some of the observed improvement, an alternative explanation may also be that a series of two nitrous oxide treatments may have additive and sustained efficacy compared to a single nitrous oxide inhalation treatment.

To put the magnitude of these effects in context, Gibbons et al. 2012 synthesized the data for 37 adult and geriatric placebo controlled double-blind randomized trials of fluoxetine and venlafaxine using the HDRS-17 as an outcome. They found an estimated separation of −2.55 HDRS-17 units at 6 weeks between active treatment and placebo control. Given the 24% increased range of the 21 item HDRS relative to the 17 item HDRS, this is equivalent to a −3.16 unit difference on the HDRS-21 at 6 weeks. Smaller effects were observed at 2 weeks (see Gibbons et. al 2012, FIG. 1). (16, 17) By contrast, we found a difference of −7.00 HDRS-21 units for 50% nitrous oxide at 2 weeks and −5.19 units for 25% nitrous oxide at 2 weeks, following a single treatment. This becomes particularly relevant in the context of the trial patient population (this study being severe TRMD, Gibbons et al., being milder depression).

Because this was a phase 2 clinical trial with the goal to determine the risk/benefit ratio for 50% versus 25% nitrous oxide for treatment-resistant major depressive disorder, one must be cautious in extrapolating the findings of a small trial to a large patient population. However, these trial results suggest that it may be reasonable to start treatment with a lower dose of nitrous oxide (25%) because of its comparable efficacy and lower risk profile but consider escalating to 50% when a stronger treatment effect is desired.

Since publication of the first trial investigating the potential use of nitrous oxide in the treatment of TRMD, one case report has shown efficacy of a single inhalation of 50% nitrous oxide beyond one month; (18) early promise in the treatment of post-traumatic stress disorder in U.S. veterans, (19) and in an experimental model of psychological trauma to simulate post-traumatic stress disorder. (20)

Discussion-Safety

While the efficacy of 25% nitrous oxide was similar to 50% nitrous oxide, the risk of adverse effects was not. Inhalation of 25% nitrous oxide compared to 50% nitrous oxide was associated with a four-fold decrease in adverse events. Nearly all adverse events occurred during or immediately after the administration of nitrous oxide and were typically limited to a few hours. Adverse events were related to sedation (e.g. sleepiness), mild dissociative effects (lightheadedness, paranoia, feeling high), and nausea and vomiting. (21, 22)

Limitations

There are several limitations of this study. First, the sample size was appropriate for a dose-finding phase 2 trial but small; however, not too small to detect significant separation between treated and control patients. Second, the follow-up was limited to 2 weeks and a single treatment. We note however, that for the entire 3-month treatment regimen, which involved 2 treatments with nitrous oxide, a median reduction of 11 HDRS-21 points was observed, which is much larger than that typically seen for traditional antidepressant trials. (16, 17) Third, despite having a 1-month interval between treatment sessions, some patients had a sustained treatment effect beyond 4 weeks which resulted in a carryover effect and influenced the study power. Fourth, in more than 4 out of 5 instances, patients correctly guessed their treatment (nitrous oxide versus placebo), which is higher than expected due to chance. Thus, there is potential for bias in the study results, although in a similar clinical trial, we did not observe a treatment effect of nitrous oxide for bothersome tinnitus despite 85% of patients correctly guessing the treatment arm. (23)

Given the calming effects of nitrous oxide (for this reason it is used in dental procedures), it is extremely difficult to completely blind patients to nitrous oxide versus placebo. Concomitant use of a relaxing agent in the placebo group (e.g., benzodiazepines) to artificially mimic the temporary euphoric/anxiolytic effects of nitrous was considered; however, out of concern that this could differentially affect depressive symptoms, we decided not to employ this method. (24) That said, there was a small placebo effect observed (FIG. 1), which appeared to peak at 24 hours and subside by 2 weeks. The fact that some patients changed the dosage or choice of their antidepressant medication may have influenced some study results. Lastly, the non-significant results of 25% nitrous oxide on QIDS and POMS scale raise the concern of functional unblinding, since the 50% nitrous oxide inhalation had markedly higher rates of adverse events and would thus have been most easily discriminated from placebo by the participants in the cross-over design.

### CONCLUSIONS

The findings of this study support that a lower concentration of nitrous oxide (25%) has similar efficacy in treatment-resistant major depression, as compared to 50% nitrous oxide, while having a markedly lower risk of adverse events. The antidepressant effects of nitrous oxide may last between two and four weeks.

### REFERENCES

1. C. Kraus, B. Kadriu, R. Lanzenberger, C. A. Zarate, Jr., S. Kasper, Prognosis and improved outcomes in major depression: a review. Transl Psychiatry 9, 127 (2019).
2. D. L. Dunner, A. J. Rush, J. M. Russell, M. Burke, S. Woodard, P. Wingard, J. Allen, Prospective, long-term, multicenter study of the naturalistic outcomes of patients with treatment-resistant depression. J Clin Psychiatry 67, 688-695 (2006).
3. A. J. Rush, S. R. Wisniewski, S. Zisook, M. Fava, S. C. Sung, C. L. Haley, H. N. Chan, W. S. Gilmer, D. Warden, A. A. Nierenberg, G. K. Balasubramani, B. N. Gaynes, M. H. Trivedi, S. D. Hollon, Is prior course of illness relevant to acute or longer-term outcomes in depressed out-patients? A STAR*D report. Psychol Med 42, 1131-1149 (2012).
4. L. Thomas, D. Kessler, J. Campbell, J. Morrison, T. J. Peters, C. Williams, G. Lewis, N. Wiles, Prevalence of treatment-resistant depression in primary care: cross-sectional data. Br J Gen Pract 63, e852-858 (2013).
5. D. S. Hasin, A. L. Sarvet, J. L. Meyers, T. D. Saha, W. J. Ruan, M. Stohl, B. F. Grant, Epidemiology of Adult DSM-5 Major Depressive Disorder and Its Specifiers in the United States. JAMA Psychiatry 75, 336-346 (2018).
6. H. A. Whiteford, L. Degenhardt, J. Rehm, A. J. Baxter, A. J. Ferrari, H. E. Erskine, F. J. Charlson, R. E. Norman, A. D. Flaxman, N. Johns, R. Burstein, C. J. Murray, T. Vos, Global burden of disease attributable to mental and substance use disorders: findings from the Global Burden of Disease Study 2010. Lancet 382, 1575-1586 (2013).
7. P. Nagele, A. Duma, M. Kopec, M. A. Gebara, A. Parsoei, M. Walker, A. Janski, V. N. Panagopoulos, P. Cristancho, J. P. Miller, C. F. Zorumski, C. R. Conway, Nitrous Oxide for Treatment-Resistant Major Depression: A Proof-of-Concept Trial. Biol Psychiatry 78, 10-18 (2015).
8. G. Sanacora, M. A. Frye, W. McDonald, S. J. Mathew, M. S. Turner, A. F. Schatzberg, P. Summergrad, C. B. Nemeroff, B. American Psychiatric Association Council of Research Task Force on Novel, Treatments, A Consensus Statement on the Use of Ketamine in the Treatment of Mood Disorders. JAMA Psychiatry 74, 399-405 (2017).
9. P. Nagele, L. B. Metz, C. M. Crowder, Nitrous oxide (N(2)O) requires the N-methyl-D-aspartate receptor for its action in Caenorhabditis elegans. Proc Natl Acad Sci USA 101, 8791-8796 (2004).
10. S. Mennerick, V. Jevtovic-Todorovic, S. M. Todorovic, W. Shen, J. W. Olney, C. F. Zorumski, Effect of nitrous oxide on excitatory and inhibitory synaptic transmission in hippocampal cultures. J Neurosci 18, 9716-9726 (1998).
11. V. Jevtovic-Todorovic, S. M. Todorovic, S. Mennerick, S. Powell, K. Dikranian, N. Benshoff, C. F. Zorumski, J. W. Olney, Nitrous oxide (laughing gas) is an NMDA antagonist, neuroprotectant and neurotoxin. Nat Med 4, 460-463 (1998).
12. M. C. Kalmoe, A. M. Janski, C. F. Zorumski, P. Nagele, B. J. Palanca, C. R. Conway, Ketamine and nitrous oxide: The evolution of NMDA receptor antagonists as antidepressant agents. J Neurol Sci 412, 116778 (2020).
13. C. F. Zorumski, P. Nagele, S. Mennerick, C. R. Conway, Treatment-Resistant Major Depression: Rationale for NMDA Receptors as Targets and Nitrous Oxide as Therapy. Front Psychiatry 6, 172 (2015).
14. C. G. Abdallah, G. Sanacora, R. S. Duman, J. H. Krystal, Ketamine and rapid-acting antidepressants: a window into a new neurobiology for mood disorder therapeutics. Annu Rev Med 66, 509-523 (2015).
15. D. V. Sheehan, Y. Lecrubier, K. H. Sheehan, P. Amorim, J. Janays, E. Weiller, T. Hergueta, R. Baker, G. C. Dunbar, The Mini-International Neuropsychiatric Interview (M.I.N.I.): the development and validation of a structured diagnostic psychiatric interview for DSM-IV and ICD-10. J Clin Psychiatry 59 Suppl 20, 22-33; quiz 34-57 (1998).
16. R. D. Gibbons, K. Hur, C. H. Brown, J. M. Davis, J. J. Mann, Benefits from antidepressants: synthesis of 6-week patient-level outcomes from double-blind placebo-controlled randomized trials of fluoxetine and venlafaxine. Arch Gen Psychiatry 69, 572-579 (2012).
17. R. D. Gibbons, C. H. Brown, K. Hur, J. Davis, J. J. Mann, Suicidal thoughts and behavior with antidepressant treatment: reanalysis of the randomized placebo-controlled studies of fluoxetine and venlafaxine. Arch Gen Psychiatry 69, 580-587 (2012).
18. P. Nagele, F. Brown, V. Bass, D. Yohanna, Prolonged Remission of Major Depressive Disorder After Single Nitrous Oxide Inhalation Treatment. Frontiers in Psychiatry 11, (2020).
19. A. Varias, P. van Roessel, M. Parsiani, M. Filippou-Frye, T. C. Neylan, P. Nagele, J. Yesavage, J. D. Clark, C. I. Rodriguez, Does Nitrous Oxide Help Veterans With Posttraumatic Stress Disorder? A Case Series. J Clin Psychiatry 81, (2020).
20. R. K. Das, A. Tamman, V. Nikolova, T. P. Freeman, J. A. Bisby, A. I. Lazzarino, S. K. Kamboj, Nitrous oxide speeds the reduction of distressing intrusive memories in an experimental model of psychological trauma. Psychol Med 46, 1749-1759 (2016).
21. P. Nagele, C. F. Zorumski, C. Conway, Exploring Nitrous Oxide as Treatment of Mood Disorders: Basic Concepts. J Clin Psychopharmacol 38, 144-148 (2018).
22. W. Buhre, N. Disma, J. Hendrickx, S. DeHert, M. W. Hollmann, R. Huhn, J. Jakobsson, P. Nagele, P. Peyton, L. Vutskits, European Society of Anaesthesiology Task Force on Nitrous Oxide: a narrative review of its role in clinical practice. Br J Anaesth 122, 587-604 (2019).
23. H. Y. Hong, O. Karadaghy, D. Kallogjeri, F. T. Brown, B. Yee, J. F. Piccirillo, P. Nagele, Effect of Nitrous Oxide as a Treatment for Subjective, Idiopathic, Nonpulsatile Bothersome Tinnitus: A Randomized Clinical Trial. JAMA Otolaryngol Head Neck Surg 144, 781-787 (2018).
24. P. van Vliet, R. C. van der Mast, M. van den Broek, R. G. Westendorp, A. J. de Craen, Use of benzodiazepines, depressive symptoms and cognitive function in old age. Int J Geriatr Psychiatry 24, 500-508 (2009).

The contents of all cited references are incorporated herein in their entirety.

Numerous modifications and variations of the present invention are possible considering the above teachings. It is therefore to be understood that within the scope of the appended claims, the invention may be practiced otherwise than as specifically described herein.

We claim:

1. A method of treating a treatment-resistant depressive disorder in a subject in need thereof, comprising administering to the subject an effective amount of an inhaled gas, the gas, comprising: 25% by weight of nitrous oxide.

2. The method of claim 1, wherein the treatment-resistant depressive disorder is selected from atypical depression, bipolar disorder, catatonic depression, depressive disorder not otherwise specified, depressive personality disorder, double depression, dysthymia, major depressive disorder, melancholic depression, minor depressive disorder, postpartum depression, post-traumatic stress disorder, psychotic major depression, recurrent brief depression, seasonal affective disorder, suicidality/acute suicide risk, and treatment-resistant major depression.

3. The method of claim 1, wherein the depressive disorder is treatment-resistant major depression (TRMD).

4. The method of claim 1, wherein the inhaled gas, further comprises: oxygen, nitrogen, xenon, or combinations thereof.

5. The method of claim 1, wherein the inhaled gas, comprises: 25% by weight nitrous oxide, 5-25% by weight xenon, and the remainder oxygen.

6. The method of claim 1, wherein the inhaled gas, comprises: 25% by weight nitrous oxide, 5-25% by weight nitrogen, and the remainder oxygen.

7. The method of claim 1, wherein the inhaled gas, comprises: 25% by weight nitrous oxide and 75% oxygen.

8. The method of claim 1, wherein the inhaled gas is administered for 60 minutes.

9. The method of claim 1, wherein the 25% by weight nitrous gas is formed just prior to inhalation, by titrating a carrier gas with nitrous oxide.

10. The method of claim 1, wherein the level of nitrous oxide in the inhaled gas is titrated up to 25% by weight of nitrous oxide during the treatment.

11. The method of claim 10, wherein the titration time is from 5-10 minutes.

12. The method of claim 1, wherein the inhaled gas is administered at least one day every seven days of treatment.

13. The method of claim 1, wherein the inhaled gas is administered at least one day every two weeks of treatment.

14. The method of claim 1, wherein the inhaled gas is administered at least one day every month of treatment.

15. The method of claim 1, wherein the patient is treated for at least 1 month.

16. A method of treating a treatment-resistant depressive disorder in a subject in need thereof, comprising:
(a) administering to the subject an effective amount of an inhaled gas, comprising: 25% by weight of nitrous oxide for a first treatment period, and
(b) administering to the subject an effective amount of an inhaled gas, comprising: 50% by weight of nitrous oxide for a second treatment period.

17. The method of claim 16, wherein the first treatment period is for at least 1 week and the second treatment period is for at least 1 week.

18. A gas container, comprising: an inhalable gas, the gas, comprising: 25% by weight nitrous oxide.

19. The gas container of claim 18, wherein the gas, further comprises: oxygen, nitrogen, xenon, or combinations thereof.

20. The gas container of claim 18, wherein the gas, comprises: 25% by weight nitrous oxide, 5-25% by weight xenon, and the remainder oxygen.

21. The gas container of claim 18, wherein the gas, comprises: 25% by weight nitrous oxide, 5-25% by weight nitrogen, and the remainder oxygen.

22. The gas container of claim 18, wherein the gas, comprises: 25% by weight nitrous oxide and 75% oxygen.
